(19) United States
(12) Patent Application Publication
(10) Pub. No.: US 2017/0173097 A1
(43) Pub. Date: Jun. 22, 2017

(54) COMPOSITIONS FOR ENHANCING DETOXIFICATION AND USES THEREOF

(71) Applicant: Specialty Nutrition Group, Inc., Boca Raton, FL (US)

(72) Inventor: Gregory T. HORN, Lighthouse Point, FL (US)

(73) Assignees: Specialty Nutrition Group, Inc., Boca Raton, FL (US); Specialty Nutrition Group, Inc., Boca Raton, FL (US)

(21) Appl. No.: 15/128,841

(22) PCT Filed: Mar. 20, 2015
(86) PCT No.: PCT/US2015/021823

§ 371 Date: Sep. 23, 2016

Related U.S. Application Data
(60) Provisional application No. 61/969,457, filed on Mar. 24, 2014.

(51) Int. Cl. A61K 36/31; A61K 36/9066; A61K 31/19

(52) U.S. Cl. CPC A61K 36/31; A61K 31/19; A61K 36/9066

(57) ABSTRACT

The present invention refers to novel compositions and to methods of using the compositions to enhance the detoxification (e.g., deactivation, metabolism, and/or excretion) of various toxins, harmful substances or other foreign substances and to treat and/or prevent diseases or disorders mediated by increased levels of such toxins, harmful substances or other foreign substances. This invention also generally relates to compositions and to methods of using the compositions to maintain and/or increase the levels of glutathione stores and/or other critical components of the Phase 2 processes.

### CROSS-REFERENCE TO RELATED APPLICATIONS

This application claims the benefit of and priority to U.S. Provisional Patent Application No. 61/969,457, filed Mar. 24, 2013. The entire contents of the foregoing application are incorporated herein by reference in their entirety.

### BACKGROUND OF THE INVENTION

The human body is routinely exposed to a wide array of foreign substances from pharmaceuticals, foods and environmental chemicals and toxins. In order to protect itself, the body uses a multistage, complex system of metabolic transformations to detoxify these substances. Proper functioning of these systems is critical to minimize damage and prevent certain chronic diseases. The liver is a key organ where various metabolic transformations occur. The review by Liska [Liska D Alternative Med Rev 1998; 3: 187-98] provides an overview of the detoxification systems.

The first phase, Phase 1, involves oxidative transformation via the cytochrome P450 enzyme system. Metabolic products coming out of these transformations may still be toxic and must be further processed. Phase 2 detoxification also occurs in the liver and is the critical metabolic process for neutralizing toxic substances coming from Phase 1 enzymes and preparing the metabolites for removal via excretion. There are several metabolic reactions that occur in phase 2 transformation. These include: glucuronidation, sulfation and amino acid conjugation. Scientifically, less is known about the Phase 2 transformations compared to Phase 1 reactions. It is known to some degree that the functioning of these detoxification systems is altered by factors such as age, gender, genetic make-up, smoking, plus other lifestyle factors. Liver function tests include testing the levels for the enzyme aspartate aminotransferase (AST) (also known as serum glutamic oxaloacetic transaminase (SGOT)) and alanine aminotransferase (ALT) (also known as serum glutamic pyruvic transaminase (SGPT)). AST is normally found in a variety of tissues including the heart, kidney, muscle, brain and liver, and released into the serum when any one of these tissues is damaged. By contrast, ALT is normally found concentrated in the liver, and is released into the bloodstream as the result of liver damage as a specific indicator.

The normal range of values for AST is about 5 to 40 units per liter of serum (the liquid part of the blood). The normal range of values for ALT is about 7 to 56 units per liter of serum. Elevated levels of these enzymes may indicate impaired liver function or injury to the liver. AST is often elevated 2 or 3:1 vs ALT in alcoholics.

Bilirubin levels are another common test of liver function. Bilirubin comes from two primary sources: 1) indirect or unconjugated bilirubin, consisting of old red blood cells removed by the spleen and sent to the liver; and 2) direct or conjugated bilirubin, where the liver donates glucuronic acid, making them water soluble for excretion. A normal range for direct bilirubin is less than 08. mg/dL of blood serum, and a normal range for total bilirubin (i.e., indirect+direct bilirubin) is 0.3 mg-1.0 mg/dL of blood serum.

Nutritional status is an important factor and there is growing knowledge that certain phytonutrients play a role in modulating both phase 1 and 2 enzymes, although this work is still in progress. An extensive array of essential vitamins and minerals are involved in all phases of detoxification and the related pathways. Those vitamins and minerals that are either antioxidants or antioxidant cofactors are particularly important in Phase 2 reactions. This group includes vitamins A, C, E, B1, B5, B12, zinc, copper, selenium, folic acid, and manganese. In addition, the nonessential (in some states conditionally essential) amino acids glycine, taurine, glutamine, ornithine, and arginine participate in conjugation reactions. Glutathione and related compounds such as cysteine, N-acetyl cysteine, methionine serve as precursors in glutathione conjugation reactions. Having an adequate supply of all of these substrates, precursors and cofactors is essential for optimal detoxification. Regular intake of adequate amounts of key substrates is especially important to persons exposed to toxicants or in persons who have suboptimal metabolic processes and/or increased detoxification requirements.

Chemical sensitivity or Multiple Chemical Sensitivity (MCS) is a condition characterized by a loss of tolerance to even low-level exposure to certain chemicals. Symptoms caused by MCS can range from inconvenient to disabling, and include low energy, loss of mental concentration, muscle pain, joint pain, headache, burning or irritated eyes, skin conditions, and breathing problems.[Reference Understanding and Accommodating People With MCS, Pamela Reed Gibson, Ph.D.].

One review of a two-phase population study showed respondents with MCS indicated cleaning agents (88.4%), pesticides (81.2%), perfumes (81.2%), vehicle exhaust (72.5%), the products used in barber shops and beauty salons (60.9%), new carpeting (53.6%), new furniture (39.1%), chlorine in drinking water (29.1%), and fresh ink (26.1%) as triggers for reactions, among other triggers such as second hand smoke. [Caress S M, and Steinemann, A C (September 2003). “A review of a two-phase population study of multiple chemical sensitivities.” “Environmental Health Perspectives. 111(12): 1490-1497. ] Metabolic detoxification of chemicals for which even low levels of exposure can cause symptoms is especially important for MCS sufferers.

Metabolism of alcohol takes place primarily in the liver, in a three-step process where alcohol (ethanol) is converted to acetaldehyde, then to acetic acid and then to acetyl-CoA, which then enters the citric acid cycle ultimately ending up as water and carbon dioxide. The first conversion step creates acetaldehyde, a highly unstable compound which rapidly forms highly toxic free radicals if not quenched by antioxidants such as ascorbic acid (Vitamin C) and Vitamin B₁ (thiamine) Short term exposure is suggested in the literature to contribute to the creation and duration of a hangover following excessive alcohol consumption. Longer term exposure of the kidney and liver to these compounds in chronic alcoholics can lead to severe damage [http://ntp.niehs.nih.gov/ntp/roc/eleventh/profiles/s001acet.pdf].

Another phase 2 pathway is the glucuronidation pathway, where glucuronic acid is attached to certain toxins as well as hormones such as estrogen to facilitate their removal by excreting them via bile into the intestinal tract. However, beta-glucuronidase, a bacterial enzyme found in the intestines, can break the bond that attaches the glucuronic acid to the toxin, allowing it to be reabsorbed back into the bloodstream and contributing to the total toxic load on the liver. Beta-glucuronidase activity can be inhibited by the administration of D-glucarate as calcium D-glucarate (Walaszek Z, Szemraj J, Narog M. Metabolism, uptake, and excretion of a D-glucaric acid salt and its potential use in cancer prevention. Cancer Detect Prey 1997; 21:178-9) According to data released from the University of Texas M.D. Anderson Cancer Center, calcium D-glucarate inhibited beta-glucuronidase by 57% in the blood, 44% in the liver, 39% in the intestines, and 37% in the lungs. [Dwivedi C, Heck W J, Downie A A, et al. Effect of calcium glucarate on beta-glucuronidase activity and glucarate content of certain vegetables and fruits. Biochem Med Metab Biol 1990;43:83-92.] Such an inhibition of beta-glucuronidase can protect the action of the glucuronidation pathway.

As stated above, establishing, maintaining, and/or enhancing a robust detoxification system is essential for good health. This invention describes compositions and uses thereof for establishing, maintaining, and/or enhancing a robust detoxification system, as is lacking in the art.

### COMPOUNDS OF THE INVENTION

The invention relates generally to compositions and to methods of using the compositions delineated herein to enhance the detoxification (e.g., deactivation, metabolism, and/or excretion) of various toxins, harmful substances or other foreign substances, and to treat and/or prevent diseases or disorders mediated by increased levels of such toxins, harmful substances or other foreign substances. This invention also generally relates to compositions and to methods of using the compositions to maintain and/or increase the levels of glutathione stores and/or other critical components of the Phase 2 processes.

In one aspect, the invention provides a composition comprising at least two of the group consisting of: a) sulforaphane, sulforaphane glucosinolate, dithioethiones, and/or cruciferous vegetable extracts; b) glucaric acid or salt thereof; c) curcumin or curcuminoid extracts; d) glutathione or glutathione precursors; e) flavanols or flavonoids; f) vitamins and/or minerals; and g) probiotics.

Calcium D-glucarate is the salt of D-glucaric acid, which is produced in small quantities by the body. Glucaric acid is also found in small quantities in fruits and vegetables e.g. grapefruit, apples, oranges and cruciferous vegetables. D-glucaric acid and a related di-lactone compound are end products of the D glucuronic acid pathway. D-glucaric acid appears to reduce cell proliferation, enhance apoptosis and regulate inflammation largely by inhibiting beta-glucuronidase. These effects may be particularly useful in detoxifying carcinogens, altering estrogen metabolism and ultimately preventing cancer [Anon. Calcium-D-glucarate. Altern Med Rev. 2002 Aug; 7(4):336-9; Abou-Issa H, Dwivedi C, Curley R W Jr, Kirkpatrick R, Koolemans-Beynen A, Engineer F N, Humphries K A, el-Masry W, Webb T E. Basis for the anti-tumor and chemopreventive activities of glucarate and the glucarate:retinoid combination. Anticancer Res. 1993 Mar-Apr;13(2):395-9; Hanausek M, Walaszek Z, Slaga T J. Detoxifying cancer causing agents to prevent cancer. Integr Cancer Ther. 2003 Jun; 2(2):139-44; Walaszek Z, Szemraj J, Narog M, Adams A K, Kilgore J, Sherman U, Hanausek M. Metabolism, uptake, and excretion of a D-glucaric acid salt and its potential use in cancer prevention. Cancer Detect Prey. 1997; 21(2):178-90; Zóltaszek R, Hanausek M, Kiliańska Z M, Walaszek Z, Postepy Hig Med Dosw (Online). 2008 Sep 5;62:451-62; Hanausek M, Walaszek Z, Slaga T J, Detoxifying cancer causing agents to prevent cancer. Integr Cancer Ther. 2003 Jun;2(2):139-44; Ahem Med Rev. 2002 Aug;7(4):336-9].

Isothiocyanates are one class of compounds found in cruciferous vegetables (e.g., broccoli, broccoli sprouts). This class of compounds has been shown in numerous laboratory studies to have high potential as cancer chemopreventive agents [Juge N, Mithren R F, Traka M. Cell Mol Life Sci 2007; 64: 1105-27; Tan X L, Spivack S D. Lung Cancer 2009; 65: 129-137]. One specific mode of action that has been deemed important is that these compounds are important inducers that promote detoxification by Phase 2 enzymes, especially via Keap1-Nrf2 signaling and antioxidant response element (ARE)-driven gene expression [Abdull Razis A F, Bagatta M, De Nicola G R, Iori R, Ioannides C. Lung Cancer. 2011 Mar; 71 (3); 298-305; Boddupalli S, Mein J R, Lakkanna S, James D R. Front Genet 2012; 3: 7; Emmert S W, Desai D, Amin S, Richie J P Jr. Bioorg Med Chem Lett. 2010 Apr 15; 20(8):2675-9; Gan N, Mi L, Sun X, Dai G, Chung F L, Song. Toxicol Appl Pharmacol 2010; 247: 129-37; Brooks J D, Paton V G, Vidanes G. Cancer Epidemiol Biomarkers Prey. 2001 Sep; 10(9):949-54; Leoncini E, Malaguti M, Angeloni C, Motori E, Fabbri D, Hrelia S. J Food Sci 2011; 76: H175-81; Lii C K, Liu K L, Cheng Y P, Lin A H, Chen H W, Tsai C W. J Nutr 2010; 140: 885-92; Matusheski N V, Jeffery EH. J Agric Food Chem 2001 49: 5743-9; Misiewicz I, Skupinska K, Kowalska E, Lubinski J, Kasprzycka-Guttman T. Acta Biochim Pol. 2004; 51: 711-21; Myzak M C, Dashwood R H. Cancer Lett 2006; 233: 208-18; Prestera T, Talalay P. Proc Natl Acad Sci USA 1995; 92: 8965-9; Razis A F, Bagatta M, De Nicola G R, Iori R, Plant N, Ioannides C. J Agric Food Chem 2012; 60: 5556-64; Robbins MG, Hauder J, Somoza V, Eshelman B K, Barnes D M, Hanlon PR. J Food Sci 2010; 75: H190-9; Wang H, Khor T O, Yang Q, Huang Y, Wu T Y, Saw C L, Lin W, Androulakis I P, Kong A N. Mol Pharm 2012; 9: 2819-27; Warwick E, Cassidy A, Hanley B, Jouni Z E, Bao Y. Br J Nutr 2012 108: 2158-65; Baek S H, Park M, Suh J H, Choi H S, Biosci Biotechnol Biochem. 2008 May;72(5):1176-82; Duan W, Li X, Shi J, Guo Y, Li Z, Li C, Neuroscience. 2010 Sep 15;169(4):1621-9; Fimognari C, Nüsse M, Berti F, Ion R, Cantelli-Forti G, Hrelia P, Ann N Y Acad Sci. 2003 Dec;1010:393-8; Fimognari C, Nüsse M, Cesari R, Ion R, Cantelli-Forti G, Hrelia P, Carcinogenesis. 2002 Apr;23(4):581-6; Jackson S J, Singletary KW, J Nutr. 2004 Sep;134(9):2229-36; Jackson S J, Singletary K W, Carcinogenesis. 2004 Feb;25(2):219-27; Kalpana Deepa Priya D, Gayathri R, Sakthisekaran D, Biomed Pharmacother. 2011 Feb;65(1):9-16; Kensler T W, Curphey T J, Maxiutenko Y, Roebuck B D, Drug Metabol Drug Interact. 2000;17(1-4):3-22; Khiar N, Werner S, Mallouk S, Lieder F, Alcudia A, Fernandez I, J Org Chem. 2009 Aug 21;74(16):6002-9; Myzak M C, Karplus P A, Chung F L, Dashwood RH, Cancer Res. 2004 Aug 15;64(16):5767-74; Pernice R, Hauder J, Koehler P, Vitaglione P, Fogliano V, Somoza V, Mol Nutr Food Res. 2009 Dec;53(12):1540-50; Perocco P, Bronzetti G, Canistro D, Valgimigli L, Sapone A, Affatato A, Pedulli G F, Pozzetti L, Broccoli M, Iori R, Barillari J, Sblendorio V, Legator M S, Paolini M, Abdel-Rahman S Z, Mutat Res. 2006 Mar 20;595(1-2):125-36; Sibhatu M B, Smitherman P K, Townsend A J, Morrow C S, Carcinogenesis. 2008 Apr;29(4):807-15; Trinh K, Moore K, Wes P D, Muchowski PJ, Dey J, Andrews L, Pallanck L J, J Neurosci. 2008 Jan 9;28(2):465-72; Veeranki O L, Bhattacharya A, Marshall J R, Zhang Y, Br J Nutr. 2013 Jan 14;109(1):25-32; Xu C, Li CY, Kong A N, Arch Pharm Res. 2005 Mar;28(3):249-68; Xu T, Ren D, Sun X, Yang G, Anticancer Agents Med Chem. 2012 Nov;12(9):1132-42; Ye L, Zhang Y, Carcinogenesis. 2001 Dec;22(12):1987-92; Zhang Y, Talalay P, Cho C G, Posner G H, Proc Natl Acad Sci U S A. 1992 Mar 15;89(6):2399-403; Zhu H, Jia Z, Strobl J S, Ehrich M, Misra H P, Li Y, Cardiovasc Toxicol. 2008 Fall;8(3):115-25]. A review by Clarke et al [Clarke J D, Dashwood RH, Ho E. Cancer Lett. 2008 Oct 8; 269(4291-304] discusses the specific studies conducted to support these modes of action. Additional evidence suggests that sulphoraphane suppresses tumor development during post-initiation by induction of cell cycle arrest and apoptosis. Some additional work suggests that sulforaphane can alter histone acetylation and affect gene regulation via inhibition of histone deacetylase enzymes [Clark J D, Riedl K, Bella D, Schwartz S J, Stevens J F, Ho E. J Agric Food Chem. 2011 Oct 26; 59(20):10955-63]. Within the class of compounds called isothiocyanates, sulfurophane has been found to have the greatest impact. Recent research suggests that additional compounds in cruciferous vegetables, dithiolethiones and dithiins, also contribute to induction of phase 2 detoxification enzymes [Kensler T W, Curphey T J, Maxiutenko Y, Roebuck B D. Drug Metabol Drug Interact. 2000; 17: 2-22]. While limited clinical work has been undertaken to assess the cancer chemoprevention effects, this area has been recently reviewed [James D, Devaraj S, Bellur P, Lakkanna S, Vicini J, Boddupalli S. Nutr Rev. 2012 Nov;70(11):654-65].

Curcumin is another natural product that holds great potential as a detoxifying and chemopreventative agent [Asher G N, Spelman K Altern Ther Health Med. 2013 Mar-Apr;19(2):20-2]. Curcumin contains a variety of constituents that may exert favorable biological reactions [Dinkova-Kostova A T. Mini Rev Med Chem. 2002 Dec;2(6):595-610]. Curcumin is known to enhance the function of Phase 2 enzymes. This activity has been demonstrated in a variety of laboratory experiments [Dinkova-Kostova A T, Talalay P. Carcinogenesis. 1999 May;20(5):911-4; Garg R, Gupta S, Maru GB. Carcinogenesis. 2008 May;29(5):1022-32; Lee J S, Surh Y J. Cancer Lett. 2005 Jun 28;224(2):171-84; Odenthal J, an Heumen B W, Roelofs H M, to Morsche R H, Marian B, Nagengast F M, Peters W H. Nutr Cancer. 2012 Aug;64(6):856-63; Iqbal M, Sharma SD, Okazaki Y, Fujisawa M, Okada S. Pharmacol Toxicol. 2003 Jan;92(1):33-8; Thimmulappa R K, Rangasamy T, Alam J, Biswal S. Med Chem. 2008 Sep;4(5):473-81]. Curcumin, while active orally, generally has low bioavailability. There are a small number of curcumin preparations now marketed or in development that employ technology that enhances their bioavailability. One such preparation that is commercially available is Meriva, which is a patented form in a lipid carrier. An overview of the material and studies that have been performed using it are described on the manufacturer's website [http://www.phytosomes.info/public/meriva.asp]. Curcumin is a pleotropic substance with a variety of actions beyond enhancement of Phase 2 enzymes. Noteworthy is its anti-inflammatory actions which may contribute to its effectiveness in detoxification and cancer prevention [Jurenka J S. Ahem Med Rev. 2009 Jun;14(2):141-53; Shehzad A, Wahid F, Lee Y S. Arch Pharm (Weinheim). 2010 Sep;343(9):489-99; Shen G, Xu C, Hu R, Jain M R, Gopalkrishnan A, Nair S, Huang MT, Chan J Y, Kong A N. Modulation of nuclear factor E2-related factor 2-mediated gene expression in mice liver and small intestine by cancer chemopreventive agent curcumin Mol Cancer Ther. 2006 Jan;5(1):39-51]. Cheung et al [Cheung K L, Khor T O, Kong A N. Pharm Res. 2009 Jan;26(1):224-31] evaluated the combination of sulphoraphane and curcumin for synergism in anti-inflammatory actions and found the combination to be effective. However, they did not explore nor suggest any synergistic activities of sulphoraphane and curcumin in Phase 2 enzyme activity or the detoxification process.

There is also some evidence that a number of tea flavonoids including theaflavin and epigallocatechin galate may be useful detoxificants and chemopreventive agents. Xu et al (2005) and Moon et al (2006) have reviewed this area [Xu C, Li CY, Kong AN. Arch Pharm Res 2005 28: 249-68; Moon Y J, Wang X, Morris M E. Toxicol In Vitro 2006: 20: 187-210]. These compounds have been shown in a number of laboratory models to enhance Phase 2 detoxification [Glei M, Pool-Zobel B L. Toxicol In Vitro. 2006; 20: 295-300; Kumaraguruparan R, Seshagiri P B, Hara Y, Naagini S. Mol Carcinog 2007; 46: 797-806; Reuland D J, Khademi S, Castle C J, Irwin D C, McCord J M, Miller B F, Hamilton K L. Free Radic Biol Med 2013; 56: 102-11; Saha P, Das S. Teratog Carcinog Mutagen. 2003; Supple 1; 313-22].

### BRIEF DESCRIPTION OF THE INVENTION

In one aspect, the invention describes a composition comprising: a) sulforaphane, sulforaphane glucosinolate, dithioethiones, and/or cruciferous vegetable extracts (preferably, sulforaphane or sulforaphane glucosinate); and b) glucaric acid or salt thereof.

In another aspect, any composition described herein may further comprise curcumin or curcuminoid extracts.

In another aspect, any composition described herein may further comprise at least one of the group consisting of: a) glutathione or glutathione precursors; b) flavanols or flavonoids; c) vitamins and/or minerals; and d) probiotics.

In any of the embodiments presented herein, glucaric acid or salt thereof is D-glucaric acid (e.g., calcium D-glucarate).

In any of the embodiments presented herein, glutathione or glutathione precursors are selected from the group consisting of L-glutamine, N-acetyl-L-cysteine, glycine, whey, and indole-3-carbynol.

In any of the embodiments presented herein, vitamins and/or minerals are selected from the group consisting of vitamin C, vitamin B1, vitamin B5, zinc, magnesium, folic acid, and vitamin B12.

In any of the embodiments presented herein, the flavanols or flavonoids are enriched or further enriched with epigallocatechin gallate (ECGC).

In another aspect, the invention provides a composition comprising a) sulforaphane or sulforaphane glucosinolate; and b) calcium D-glucarate. In another aspect, sulforaphane or sulforaphane glucosinolate is about 5-300 mg. In another aspect, calcium D-glucarate is about 500-5000 mg.

In another aspect, the invention provides a composition comprising a) sulforaphane or sulforaphane glucosinolate; b) curcumin or curcuminoid extracts; and c) calcium D-glucarate.

In another aspect, sulforaphane or sulforaphane glucosinolate is about 5-300 mg. In another aspect, curcumin or curcuminoid extracts is about 100-2000 mg. In another aspect, calcium D-glucarate is about 500-5000 mg.

In another aspect, the invention provides a composition comprising a) sulforaphane or sulforaphane glucosinolate; b) curcumin or curcuminoid extracts; c) glucaric acid or salt thereof; and d) glutathione or glutathione precursors. In another aspect, sulforaphane or sulforaphane glucosinolate is about 5-300 mg. In another aspect, curcumin or curcuminoid extracts is about 100-2000 mg. In another aspect, glucaric acid or salt thereof is about 500-5000 mg calcium D-glucarate. In another aspect, glutathione or glutathione precursors comprise about 25-1500 mg L-glutamine, 25-1500 mg N-acetyl-L-cycteine, and/or 25-1500 mg glycine. In another aspect, glutathione or glutathione precursors comprise about 25-1500 mg L-glutamine, 25-1500 mg N-acetyl-L-cycteine, 25-1500 mg glycine, and/or 20-100 mg indole-3-carbynol.

In any of the embodiments presented herein, sulforaphane or sulforaphane glucosinolate is isolated, extracted, or concentrated from cruciferous vegetables or from a synthetic source.

In any of the embodiments presented herein, curcumin or curcuminoid extracts is isolated, extracted, or concentrated from turmeric or from a synthetic source.

In any of the embodiments presented herein, flavanols and/or flavonoids are isolated, extracted, or concentrated from cocoa, chocolate, or tea or from a synthetic source (preferably from tea).

In any of the embodiments presented herein, sulforaphane or sulforaphane glucosinolate is isolated, extracted, or concentrated from broccoli seeds, plants or sprouts.

In another aspect, this invention relates to a multicomponent formula. The components each have a specific mode of action in the body, and their detoxification effects are complimentary. The components each have their own characteristic bioavailability and metabolism. It is important to note that the compositions covered in this invention optimize effectiveness by combining components with complementary actions. Components of the formulas also have different pharmacokinetic properties. Combining select components enhances their overall effectiveness. Combining these components also allows one product to offer a broad approach to managing the complex detoxification process.

In another aspect, the invention provides a method of maintaining or increasing the levels of glutathione stores in a subject comprising administering to said subject an effective amount of any composition delineated herein, such that said glutathione stores are maintained or increased. In another aspect, the glutathione stores are maintained or increased by enhancing Phase 2 metabolism.

In another aspect, the invention provides a method of enhancing phase 2 metabolism in a subject comprising administering to said subject an effective amount of any composition delineated herein, such that said phase 2 metabolism is enhanced.

In another aspect, the invention provides a method of treating chemical sensitivity in a subject comprising administering to said subject an effective amount of any composition delineated herein, such that said chemical sensitivity is reduced.

In another aspect, the invention provides a method of treating toxic effects of exposure to environmental toxicants and/or poisons in a subject comprising administering to said subject an effective amount of any composition delineated herein, such that said toxic effects are reduced.

In another aspect, the invention provides a method of treating a proliferative disease in a subject comprising administering to said subject an effective amount of any composition delineated herein, such that said subject is treated for said proliferative disease. In another aspect, the proliferative disease is cancer. In another aspect, the cancer is hormone dependent. In another aspect, the hormone-dependent cancer is estrogen-positive breast cancer. In another aspect, the estrogen-positive breast cancer is classified by the marker CA2729.

In another aspect, any of the embodiments presented may further comprise one or more additional anti-cancer therapies. In another aspect, said additional anti-cancer therapy is selected from the group consisting of chemotherapy, anti-hormonal therapy, hormone receptor inhibitors, and surgical intervention.

In another aspect, the invention provides a method of improving and/or maintaining liver function in a subject comprising administering to said subject an effective amount of any composition delineated herein, such that said liver function is increased. In certain aspects, the subject possesses normal liver function prior to administration of said composition. In certain aspects, the subject possesses impaired liver function prior to administration of said composition.

In another aspect, the invention provides a method of treating acute or chronic liver impairment in a subject comprising administering to said subject an effective amount of any composition delineated herein, such that said liver impairment is decreased. In certain aspects, the acute or chronic liver impairment is due to disease or exposure to toxicants. In certain aspects, the acute or chronic liver impairment is due to exposure to acetaminophen or ethanol.

In another aspect, the invention provides a method of reducing the duration and/or severity of the central nervous system effects (CNS) of a substance that is metabolized and/or excreted via one or more phase 2-dependent pathways in a subject comprising administering to said subject an effective amount of any composition delineated herein, such that said CNS effects are decreased. In certain aspects, the substance is ethanol. In certain aspects, the administration of said composition occurs prior to ethanol consumption. In certain aspects, the administration of said composition occurs subsequent to ethanol consumption.

In another aspect, the invention provides a method of increasing the rate of excretion and/or metabolism of a substance that is metabolized and/or excreted via one or more phase 2-dependent pathways in a subject comprising administering to said subject an effective amount of any composition delineated herein, such that levels of said substance in said subject are decreased. In certain aspects, the substance is acetaminophen or ethanol. In certain aspects, the administration of said composition occurs prior to acetaminophen or ethanol consumption. In certain aspects, the administration of said composition occurs subsequent to acetaminophen or ethanol consumption. In certain aspects, the ethanol levels are assessed by blood measurement or breathalyzer measurement. In certain aspects, the method reduces the duration and/or severity of the central nervous system effects (CNS) of ethanol. In certain aspects, the method reduces the duration and/or severity of ethanol-induced hangovers.

In another aspect, the invention provides a method of treating dependency on and/or addiction to a substance that is metabolized and/or excreted via one or more phase 2-dependent pathways in a subject comprising administering to said subject an effective amount of any composition delineated herein, such that said subject is treated for such dependency and/or addiction. In certain instances, the substance is ethanol.

In certain embodiments, the subject is a mammal, preferably a primate or human.

In another embodiment, the invention provides a method as described above, wherein the effective amount of the compound or composition (e.g., any composition delineated herein) ranges from about 0.005 μg/kg to about 500 mg/kg, preferably about 0.1 mg/kg to about 500 mg/kg, more preferably about 10 mg/kg to about 500 mg/kg of body weight.

In other embodiments, the invention provides a method as described above wherein the effective amount of the compound or composition (e.g., any composition delineated herein) ranges from about 1.0 nM to about 500 μM. In another embodiment, the effective amount ranges from about 100 nM to about 100 μM.

In other embodiments, the invention provides a method as described above wherein the effective amount of the compound or composition (e.g., any composition delineated herein) ranges from about 0.1 mg/ml to about 1000 mg/ml. In certain embodiments, the effective amount ranges from about 1.0 mg/ml to about 500 mg/ml. In another embodiment, the effective amount ranges from about 1.0 mg/ml to about 100 mg/ml.

In another embodiment, the invention provides a method as described above, wherein the compound or composition (e.g., any composition delineated herein) is administered intravenously, intramuscularly, subcutaneously, intracerebroventricularly, orally or topically.

Another object of the present invention is the use of a compound or composition (e.g., any composition delineated herein) as described herein for use in maintaining or increasing the levels of glutathione stores; enhancing phase 2 metabolism; treating chemical sensitivity; treating toxic effects of exposure to environmental toxicants and/or poisons; treating a proliferative disease or disorder (e.g., preferably, cancer; more preferably, hormone-dependent cancers; still more preferably, estrogen-positive breast cancer; most preferably, estrogen-positive breast cancer classified by the marker CA2729); improving liver function;

treating acute or chronic liver impairment; reducing the duration and/or severity of the central nervous system effects (CNS) of a substance that is metabolized and/or excreted via one or more phase 2-dependent pathways (e.g., preferably, ethanol); increasing the rate of excretion and/or metabolism of a substance that is metabolized and/or excreted via one or more phase 2-dependent pathways (e.g., preferably, acetaminophen or ethanol); reducing the duration and/or severity of ethanol-induced hangovers; and treating dependency on and/or addiction to a substance that is metabolized and/or excreted via one or more phase 2-dependent pathways (e.g., preferably, ethanol). Another object of the present invention is where the disease or disorder includes proliferative diseases (e.g., preferably, cancer; more preferably, hormone-dependent cancers; still more preferably, estrogen-positive breast cancer; most preferably, estrogen-positive breast cancer classified by the marker CA2729); liver function impairment (e.g., cirrhosis, hepatitis, fatty liver disease, and the like); and/or alcohol or drug addiction.

In one aspect, the invention provides a kit comprising an effective amount of a compound or composition (e.g., any composition delineated herein), in unit dosage form, together with instructions for administering the compound or composition thereof to a subject for maintaining or increasing the levels of glutathione stores; enhancing phase 2 metabolism; treating chemical sensitivity; treating toxic effects of exposure to environmental toxicants and/or poisons; treating a proliferative disease or disorder (e.g., preferably, cancer; more preferably, hormone-dependent cancers; still more preferably, estrogen-positive breast cancer; most preferably, estrogen-positive breast cancer classified by the marker CA2729); improving liver function; treating acute or chronic liver impairment; reducing the duration and/or severity of the central nervous system effects (CNS) of a substance that is metabolized and/or excreted via one or more phase 2-dependent pathways (e.g., preferably, ethanol); increasing the rate of excretion and/or metabolism of a substance that is metabolized and/or excreted via one or more phase 2-dependent pathways (e.g., preferably, acetaminophen or ethanol); reducing the duration and/or severity of ethanol-induced hangovers; and treating dependency on and/or addiction to a substance that is metabolized and/or excreted via one or more phase 2-dependent pathways (e.g., preferably, ethanol).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

### DETAILED DESCRIPTION

### Definitions

In order that the invention may be more readily understood, certain terms are first defined here for convenience.

As used herein, the term “at risk” of developing a certain disease or disorder (e.g., diabetes or cardiovascular disease) encompasses possessing any risk factor, susceptibility, or predisposition of developing a certain disease.

As used herein, the term “treating” a disorder encompasses preventing, ameliorating, mitigating and/or managing the disorder and/or conditions that may cause the disorder. The terms “treating” and “treatment” refer to a method of alleviating or abating a disease and/or its attendant symptoms. In accordance with the present invention “treating” includes preventing, blocking, inhibiting, attenuating, protecting against, modulating, reversing the effects of and reducing the occurrence of e.g., the harmful effects of a disorder.

As used herein, “inhibiting” encompasses preventing, reducing and halting progression.

As used herein, “activating” encompasses permitting, increasing and enhancing progression.

As used herein, “enriched” encompasses greater or increased amounts of a material or desired or active compound or agent relative to its natural or other reference state.

As used herein, as “extract” is a preparation of constituents of a material (e.g., seaweed), including for example, solvent extracts, concentrated forms of said constituents, concentrated solvent extracts, isolated chemical compounds or mixtures thereof.

The term “modulate” refers to increases or decreases in the activity of a cell in response to exposure to a compound of the invention.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and a-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of compositions presented herein are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The term “therapeutically effective amount” refers to that amount of the compound being administered sufficient to prevent development of or alleviate to some extent one or more of the symptoms of the condition or disorder being treated.

A therapeutically effective amount of compound (i.e., an effective dosage) may range from about 0.005 μg/kg to about 1000 mg/kg, preferably about 0.1 mg/kg to about 1000 mg/kg, more preferably about 10 mg/kg to about 500 mg/kg of body weight. In other embodiments, the therapeutically effective amount may range from about 0.10 nM to about 500 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. It will also be appreciated that the effective dosage of a compound used for treatment may increase or decrease over the course of a particular treatment.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. In aspects, the compounds of the invention are prodrugs of any of the formulae herein.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the compounds of the invention include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

### Compounds of the Invention

Compounds (e.g., isolated compounds, compounds within extracts, compounds fractionated from extracts) of the invention can be made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jahnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

The present invention also contemplates solvates (e.g., hydrates) of a compound of herein, compositions thereof, and their use in the treatment and/or prevention of inflammatory- or reactive oxygen species (ROS)-mediated diseases. As used herein, “solvate” refers to the physical association of a compound of the invention with one or more solvent or water molecules, whether organic or inorganic. In certain instances, the solvate is capable of isolation, for example, when one or more solvate molecules are incorporated in the crystal lattice of the crystalline solid.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

### Pharmaceutical Compositions

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic (or unacceptably toxic) to the patient.

In use, at least one compound according to the present invention is administered in a pharmaceutically effective amount to a subject in need thereof in a pharmaceutical carrier by intravenous, intramuscular, subcutaneous, or intracerebroventricular injection or by oral administration or topical application. In accordance with the present invention, a compound of the invention may be administered alone or in conjunction with a second, different therapeutic. By “in conjunction with” is meant together, substantially simultaneously or sequentially. In one embodiment, a compound of the invention is administered acutely. The compound of the invention may therefore be administered for a short course of treatment, such as for about 1 day to about 1 week. In another embodiment, the compound of the invention may be administered over a longer period of time to ameliorate chronic disorders, such as, for example, for about one week to several months depending upon the condition to be treated.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific organozinc compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

A decided practical advantage of the present invention is that the compound may be administered in a convenient manner such as by intravenous, intramuscular, subcutaneous, oral or intra-cerebroventricular injection routes or by topical application, such as in creams or gels. Depending on the route of administration, the active ingredients which comprise a compound of the invention may be required to be coated in a material to protect the compound from the action of enzymes, acids and other natural conditions which may inactivate the compound. In order to administer a compound of the invention by other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound of the invention in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

Topical administration of the pharmaceutical compositions of this invention is especially useful when the desired treatment involves areas or organs readily accessible by topical application. For topical application topically to the skin, the pharmaceutical composition should be formulated with a suitable ointment, lotion, or cream containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

For topical administration, the active compound(s), extracts, enriched extracts, or prodrug(s) can be formulated as solutions, gels, ointments, creams, suspensions, and the like.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The present invention will now be demonstrated using specific examples that are not to be construed as limiting.

### Example 1

### Compositions Presented Herein Treat Chemical Sensitivity

A male subject in his mid-40s suffering from chemical sensitivity was administered a composition of calcium D-Glucarate 1,000 mg and sulphoraphane glucosinolate 30 mg in the form of oral supplements twice daily for 30 days. After 30 days of administration, the subject had experienced significant improvement in the chemical sensitivity symptoms, and reported significantly reduced irritation from exposure to fumes such as carpet and gasoline, greater energy and reduction of sluggishness in the morning, better sleep, greater clarity of mind, higher tolerance of exposure to everyday fragrances and smells, and less stiffness in muscles and joints. These results demonstrate that the compositions presented herein are successful in treating chemical sensitivity, the symptoms of which are known to be correlated with low levels of environmental or foreign chemicals and, upon clearance (e.g., metabolism, excretion, etc.) of these chemicals (e.g., Phase 2 metabolism), patients symptoms often improve. Therefore, improvement of the aforementioned subject's chemical sensitivity symptoms strongly suggest that the compositions presented herein accelerate the clearance (i.e., Phase 2 metabolism) of the chemical and/or environmental toxicants (i.e., the source of the chemical sensitivity symptoms).

### Example 2

### Compositions Presented Herein Improve Cancer Markers

A female subject in her mid-70's suffering from estrogen-positive breast cancer that had metastasized to her femur bone was administered a composition of calcium D-Glucarate 1,000 mg, sulphoraphane glucosinolate 30 mg, and turmeric complex 500 mg in the form of oral supplements twice daily for 150 days. For at least 30 days prior to the administration of the composition, the subject had been taking the drug Exemestane 25 mg daily, an aromatase inhibitor, as prescribed by her physician, and continued this prescription throughout the period during which the supplement composition was administered. After 30 days of administration, the subject experienced significant improvement in cancer markers, specifically the CA2729 marker, from 815 in December 2012 to 714 in January 2013. After 60 days of administration, the CA2729 marker had decreased to 505 in February 2013, and continued to decline to 368 in March 2013 after 90 days of administration and 256 in May 2013 after 150 days of administration. The subject also noticed that tumors had reduced noticeably in size during this time period. The subject continued to take the composition beyond the 150 days, and had a CA2729 reading of 198 in October 2013.

All patents, patent applications, and published references cited herein are hereby incorporated by reference in their entirety.

While this invention has been particularly illustrated and described with reference to particular examples, it will be understood by those skilled in the art that various changes in form and details may be made therein without departing from the scope and spirit of the invention encompassed by the appended claims.

1. A composition comprising: a) sulforaphane, sulforaphane glucosinolate, dithioethiones, and/or cruciferous vegetable extracts; and b) glucaric acid or salt thereof.

2. The composition of claim 1 comprising: a) sulforaphane or sulforaphane glucosinate; and b) glucaric acid or salt thereof.

3. The composition of claim 2, further comprising curcumin or curcuminoid extracts.

4. The composition of claim 1 further comprising at least one of the group consisting of: a) glutathione or glutathione precursors; b) flavanols or flavonoids; c) vitamins and/or minerals; and d) probiotics.

5. The composition of claim 4, wherein a) glucaric acid or salt thereof is D-glucaric acid; b) glutathione or glutathione precursors are selected from the group consisting of L-glutamine, N-acetyl-L-cysteine, glycine, whey, and indole-3-carbynol; and c) vitamins and/or minerals are selected from the group consisting of vitamin C, vitamin B1, vitamin B5, zinc, magnesium, folic acid, and vitamin B12.

6. The composition of claim 5, wherein the D-glucaric acid is in the form of calcium D-glucarate.

7. The composition of claim 5, wherein the flavanols or flavonoids are enriched with epigallocatechin gallate (ECGC).

8. The composition of claim 1, wherein:
a. sulforaphane or sulforaphane glucosinolate is about 5-300 mg; and
b. calcium D-glucarate is about 500-5000 mg.

9. The composition of claim 1, wherein:
a. sulforaphane or sulforaphane glucosinolate is about 5-300 mg;
b. curcumin or curcuminoid extracts is about 100-2000 mg; and
c. calcium D-glucarate is about 500-5000 mg.

10. The composition of claim 4, wherein:
a. sulforaphane or sulforaphane glucosinolate is about 5-300 mg;
b. curcumin or curcuminoid extracts is about 100-2000 mg;
c. glucaric acid or salt thereof is about 500-5000 mg calcium D-glucarate; and
d. glutathione or glutathione precursors comprise about 25-1500 mg L-glutamine, 25-1500 mg N-acetyl-L-cycteine, and/or 25-1500 mg glycine.

11. The composition of claim 4, wherein:
a. sulforaphane or sulforaphane glucosinolate is about 5-300 mg;
b. curcumin or curcuminoid extracts is about 100-2000 mg;
c. glucaric acid or salt thereof is about 500-5000 mg calcium D-glucarate; and
d. glutathione or glutathione precursors comprise about 25-1500 mg L-glutamine, 25-1500 mg N-acetyl-L-cycteine, 25-1500 mg glycine, and/or 20-100 mg indole-3-carbynol.

12. The composition of claim 4, wherein
a. sulforaphane or sulforaphane glucosinolate is isolated, extracted, or concentrated from cruciferous vegetables or a synthetic source;
b. curcumin or curcuminoid extracts is isolated, extracted, or concentrated from turmeric or a synthetic source;
c. flavanols and/or flavonoids are isolated, extracted, or concentrated from cocoa, chocolate, or tea or a synthetic source; and
d. sulforaphane or sulforaphane glucosinolate is isolated, extracted, or concentrated from broccoli seeds, plants or sprouts.

13. A method of maintaining or increasing the levels of glutathione stores in a subject comprising administering to said subject an effective amount of a composition according to claim 2, such that said glutathione stores are maintained or increased.

14. The method of claim 13, wherein the glutathione stores are maintained or increased by enhancing Phase 2 metabolism in said subject.

15. (canceled)

16. (canceled)

17. (canceled)

18. A method of treating a proliferative disease in a subject comprising administering to said subject an effective amount of a composition according to claim 2, such that said subject is treated for said proliferative disease.

19. The method of claim 18, wherein the proliferative disease is cancer.

20. The method of claim 19, wherein the cancer is hormone dependent.

21. The method of claim 20, wherein the hormone-dependent cancer is estrogen-positive breast cancer.

22. The method of claim 21, wherein the estrogen-positive breast cancer is classified by the marker CA2729.

23. The method of claim 18, wherein said method further comprises one or more additional anti-cancer therapies.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)11

43. (canceled)
